(19) United States
(12) Patent Application Publication
(10) Pub. No.: US 2024/0277790 A1
(43) Pub. Date: Aug. 22, 2024

(54) RECOMBINANT BACULOVIRUSES, ALPHAVIRUS VIRUS-LIKE REPLICON PARTICLES PRODUCED THEREOF AND THEIR USES

(71) Applicant: National Defense Medical Center, Taipei City (TW)

(72) Inventors: Hui-Chung LIN, Taipei City (TW); Szu-Cheng KUO, Taipei City (TW); Der-Jiang CHIAO, Taipei City (TW); Chang-Chi LIN, Taipei City (TW); Yu-Lin HSU, Taipei City (TW); Hui-Tsu LIN, Taipei City (TW); Shan-Ko TSAI, Taipei City (TW)

(73) Assignee: National Defense Medical Center, Taipei City (TW)

(21) Appl. No.: 18/172,359

(22) Filed: Feb. 22, 2023

(51) Int. Cl. A61K 35/76; C12N 7/00; C12N 15/86

(52) U.S. Cl. CPC A61K 35/76; C12N 7/00; C12N 15/86; C12N 2770/36143

(57) ABSTRACT

Disclosed herein is a recombinant baculovirus for producing an alphavirus virus-like replicon particle (VRP) having an exogenous gene in a mosquito cell. Said recombinant baculovirus comprises: (1) a replicon comprising a first promoter, and a first polynucleotide encoding at least one alphavirus non-structural protein and is operably linked to the first promoter; and (2) a helper comprising a second promoter, and a second polynucleotide encoding at least one alphavirus structural protein and is operably linked to the second promoter; wherein, the helper is upstream or downstream to the replicon; and the first promoter is less effective than the second promoter in driving gene expression in the mosquito cell. Also encompassed in the present disclosure are methods for detecting an antibody against an alphavirus in a biological sample, and/or screening an antiviral agent suitable for treating an alphavirus infection, with the aid of the alphavirus VRP produced by the present recombinant baculovirus.

### SEQUENCE LISTING XML

The present application is being filed along with a Sequence Listing XML in electronic format. The Sequence Listing XML is provided as a XML file entitled “P4225-US_SeqList_20230217_filed.xml,” created Jan. 12, 2023, which is 6 Kb in size. The information in the electronic format of the Sequence Listing XML is incorporated herein by reference in its entirety.

### BACKGROUND OF THE INVENTION

### 1. Field of the Invention

The present disclosure in general relates to the field of a recombinant baculovirus. More particularly, the present disclosure relates to a recombinant baculovirus expressing an alphavirus virus-like replicon particle (VRP), such as a CHIKV-like replicon particle.

### 2. Description of Related Art

Alphavirus is a genus of RNA viruses, with a positive-sense, single-stranded RNA genome, and is the only one genus in the Togaviridae family. It has been reported that some of the alphaviruses may infect both vertebrates and arthropods, also known as dual-host alphaviruses and arboviruses, resulting health and economic loss to humans when such alphaviruses infect humans or economic animals such as fish, birds, horses, etc. Among the insect vectors, mosquitoes are responsible for spreading terrestrial alphavirus infections; and alphavirus evolution with viral glycoprotein mutants further exacerbates arthropod-borne vector competency, leading to global spreading. Symptoms for alphavirus infection are commonly manifested as infectious arthritis, encephalitis, rashes, and fever.

Although alphavirus infection may cause considerable health hazards to humans, measures to deal with the medical issues are scant. Diagnosis for alphavirus infection remains on the reliance of identifying alphavirus from clinical samples via serological tests and/or PCR. To date, no alphavirus vaccines or antiviral agents have been approved, nor are available on the market to combat the alphavirus infection, partly due to the biosafety concerns, as alphavirus need to be handled in biosafety level 3 (BSL-3) laboratory, thereby limiting the alphavirus research. It has been proposed recently that a safe surrogate (i.e., an alphavirus VRP) may act as an alternative in the alphavirus research; however, existing method for producing the safe surrogate (i.e., co-electroporation of the in vitro-transcribed replicon and helper RNAs into mammalian cells) is not efficient to produce sufficient amount of the alphavirus VRP, and besides, uncontrolled expression of alphavirus non-structural protein 2 (nsP2) or capsid protein may render cytopathic effect (CPE) in mammalian cells, in turn limiting the production of the alphavirus VRP.

In view of the foregoing, there exists in the related art a need for an improved method for producing the alphavirus VRP, which may be generated in a sufficient amount for use in alphaviruses research, the manufacture of a vaccine or a detection reagent, or as a platform for screening drugs, so as to facilitate the prevention, the diagnosis, and/or the treatment of alphavirus infection.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

Accordingly, the present disclosure provides an improved method for producing the alphavirus VRP in mosquito cells and a recombinant baculovirus is constructed for this purpose. Such alphavirus VRP are infectious but propagation-defective, i.e., the virus cannot propagate beyond the host cell, which the particles are initially infected.

As such, as embodied and broadly described herein, one aspect of the present disclosure is directed to a recombinant baculovirus capable of producing an alphavirus VRP in a mosquito cell. Said recombinant baculovirus comprises,
a replicon comprising,
   a first promoter; and
   a first polynucleotide operably linked to the first promoter encoding at least one alphavirus non-structural protein; and
a helper comprising,
   a second promoter; and
   a second polynucleotide operably linked to the second promoter encoding at least one alphavirus structural protein;
      wherein,
the helper is upstream or downstream to the replicon; and
the first promoter is less efficient than the second promoter in driving gene expression in the mosquito cell.

According to some embodiments of the present disclosure, the first promoter is less efficient (<10⁻²) than the second promoter in driving gene expression in the mosquito cell.

According to the embodiments of the present disclosure, the first promoter may be a promoter of Ac5 gene, a promoter of ADH1 gene, a promoter of human 6-actin gene, a promoter of CAG gene, a promoter of CaMKIIa gene, a promoter of CaMV35S gene, a promoter of CMV gene, a promoter of EF1a gene, a promoter of GAL1 gene, a promoter of GAL10 gene, a promoter of GDS gene, a promoter of H1 gene, a promoter of PGK1 gene, a promoter of polyhedrin gene, a promoter of SV40 gene, a promoter of TEF1 gene, a promoter of TRE gene, a promoter of U6 gene, a promoter of UAS gene, a promoter of Ubc gene, or a promoter of Ubi gene.

Preferably, the first promoter suitable for use in the present recombinant baculovirus is the promoter of CMV gene.

Without bound to the theory, the second promoter that may be used in the present recombinant baculovirus is at least one promoter selected from the group consisting of, a promoter of the Heliothis zea Nudivirus-1 (HzNV-1) viral early expressing gene pag1, a promoter of the ceropin gene b1, a promoter of the defensin gene a4, a promoter of the heat shock protein 70 gene (hsp70), and a promoter of the homologous region 1 gene (hr1). According to one preferred example of the present disclosure, the second promoter is a composite promoter comprising the promoter of hr1 and the promoter of pag1.

According to the embodiments of the present disclosure, the at least one alphavirus non-structural protein may be non-structural protein 1 (nsP1), non-structural protein 2 (nsP2), non-structural protein 3 (nsP3), non-structural protein 4 (nsP4), and a combination thereof (e.g., any one of nsP1/nsP2, nsP1/nsP3, nsP1/nsP4, nsP2/nsP3, nsP2/nsP4, nsP3/nsP4, nsP1/nsP2/nsP3, nsP1/nsP2/nsP4, nsP1/nsP3/nsP4, nsP2/nsP3/nsP4, or nsP1/nsP2/nsP3/nsP4 protein combinations). In one working example, the at least one alphavirus non-structural protein comprises the combination of nsP1, nsP2, nsP3, and nsP4.

Also, the at least one alphavirus structural protein is selected from the group consisting of capsid (C), envelope 3 (E3), envelope 2 (E2), 6K protein (6K), envelope 1 (E1), and a combination thereof (e.g., any one of C/E3, C/E2, C/6K, C/E1, E3/E2, E3/6K, E3/E1, E2/6K, E2/E1, 6K/E1, C/E3/E2, C/E3/6K, C/E3/E1, C/E2/6K, C/E2/E1, C/6K/E1, E3/E2/6K, E3/E2/E1, E3/6K/E1, E2/6K/E1, C/E3/E2/6K, C/E3/E2/E1, C/E3/6K/E1, C/E2/K6/E1, E3/E2/6K/E1, or C/E3/E2/6K/E1 protein combinations). In one working example, the at least one alphavirus structural protein comprises the combination of C, E3, E2, 6K, and E1.

According to some advanced embodiments of the present disclosure, the present recombinant baculovirus may express exogenous gene. In such case, the present recombinant baculovirus further comprises,
a subgenomic promoter operably linked to the first polynucleotide; and
an exogenous gene operably linked to the subgenomic promoter.

In some applicable embodiments, the exogenous gene may encode a reporter protein, an expression tag, or a combination thereof.

Exemplary reporter protein may be blue fluorescence protein (BFP), cyan fluorescent protein (CFP), green fluorescence protein (GFP), enhanced green fluorescence protein (eGFP), Discosoma sp. red fluorescent protein (DsRed), yellow fluorescent proteins (YFP), enhanced yellow fluorescent proteins (eYFP), Anemonia majano fluorescent protein (amFP), Clavularia fluorescent protein (cFP), Discosoma fluorescent protein (dsFP), Zoanthus fluorescent protein (zFP), β-galactosidase (lacZ), chloramphenicol acetyltransferase (CAT), luciferase (Luc), β-lactamase, aminoglycoside-3′-phosphotransferase (APH(3′)), orotidine-5′-phosphate decarboxylase (ODCase), chitin binding protein (CBP), maltose binding protein (MBP), glutathione-S-transferase (GST), thioredoxin (TRX), alkaline phosphatase (AP), biotin-carboxy carrier protein (BCCP), calmodulin binding peptide (CBP), or a combination thereof. According to one working example of the present disclosure, the reporter protein is a combination of eGFP and Luc.

Alternatively, the exogenous gene may encode an expression tag, such expression tag as FLAG-tag, hemagglutinin (HA)-tag, poly-histidine tag, MYC-tag, NE-tag, Spot-tag, Strep-tag, or bacteriophage T7 epitope (T7-tag).

According to the embodiments of the present disclosure, the baculovirus suitable for use in the present invention is any of Autographa californica multiple nucleopolyhedrovirus (AcMNPV), Anagrapha falcifera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera exigua MNPV (SeMNPV), Spodoptera frugiperda MNPV (SfMNPV), or Trichoplusia ni MNPV (TnMNPV). In one preferred embodiment, the baculovirus is AcMNPV.

According to the embodiments of the present disclosure, the alphavirus is selected from the group consisting of Barmah Forest virus (BFV), Chikungunya virus (CHIKV), Eastern equine encephalitis virus (EEEV), Eilat virus (EILV), Everglades virus (EVEV), Middelburg virus (MIDV), O'nyong'nyong virus (ONNV), Rio Negro virus (RNV), Ross River virus (RRV), Salmon pancreas disease virus (SPDV), Semliki Forest virus (SFV), Sindbis virus (SINV), Venezuelan equine encephalitis virus (VEEV), and Western equine encephalitis virus (WEEV). In one working example, the alphavirus is CHIKV.

According to the embodiments of the present disclosure, the mosquito cell that may be transduced by the present recombinant baculovirus may be a cell derived from Aedes albopictus, Aedes aegypti, Aedes pseudoscutellaris, Anopheles sinensis, Armigeres subalbatus, Culex quinquefasciatus, Culex tritaeniorhynchus, or Toxorhynchites amboinensis. Preferably, the mosquito cell is the cell derived from Aedes pseudoscutellaris or Aedes albopictus.

As such, it would be understood that the alphavirus VRP produced by the present recombinant baculovirus serving as a vaccine to prevent an alphavirus infection is also encompassed within the scope of the present disclosure.

Another aspect of the present disclosure pertains to a method for detecting an antibody against an alphavirus in a biological sample by using an alphavirus VRP produced by the present recombinant baculovirus. Said method comprises the steps of,
(a) mixing the biological sample with an alphavirus VRP produced by the present recombinant baculovirus; and
(b) detecting a complex formed between the alphavirus VRP and the antibody against the alphavirus in the biological sample in an immunological assay,
   wherein,
the formation of the complex indicates the presence of the antibody against the alphavirus in the biological sample.

According to some preferred embodiments, the alphavirus is CHIKV.

In yet another aspect of the present disclosure, the present invention provides a method for screening an antiviral agent suitable for treating an alphavirus infection with the aid of an alphavirus VRP produced by the present recombinant baculovirus. Specifically, the method comprises,
(a) mixing a drug candidate with an alphavirus VRP produced by the present recombinant baculovirus to produce a mixture; and
(b) applying the mixture of step (a) to a mammalian cell,
   wherein,
the drug candidate is the antiviral agent if the amount of the alphavirus VRP present in the mammalian cell of step (b) is lower than that of a control, which is the mammalian cell treated with the alphavirus VRP produced by the present recombinant baculovirus alone.

According to some preferred embodiments, the alphavirus is CHIKV.

Many of the attendant features and advantages of the present disclosure will becomes better understood with reference to the following detailed description considered in connection with the accompanying drawings.

### BRIEF DESCRIPTION OF THE DRAWINGS

These and other features, aspects and advantages of the present invention will become better understood with reference to the following description, appended claims and the accompanying drawings, where:

FIGS. 1A-1B illustrate the construction and characterization of the recombinant baculovirus of Example 1, in which FIG. 1A is a schematic diagram of the recombinant baculovirus vector of Example 1; FIG. 1B are the western blot analyses of CHIKV nsP1, capsid, E2, E1 and eGFP with proteins extracted from AP-61 cells transduced with the recombinant baculovirus vector of Example 1 or with the mock control. T2A, Thosea asigna virus 2A self-cleaving peptide; HDR, hepatitis delta virus ribozyme.

FIGS. 2A-2B depict the infection of the CHIK-VRPs produced by the recombinant baculovirus of Example 1 in mosquito cells (the “mos-CHIK-VRPs”) on Vero cells. FIG. 2A are the western blot analyses of CHIKV nsP1, capsid, and eGFP with proteins extracted from Vero cells infected with the mos-CHIK-VRPs or with the mock control. FIG. 2B is the bar diagram depicting the viral RNA amplification in Vero cells infected with the mos-CHIK-VRPs. ****, p<0.0001.

FIGS. 3A-3B illustrate the kinetics of the mos-CHIK-VRPs in infecting Vero cells, in which FIG. 3A is the bar chart depicting the luciferase activity in Vero cells infected with the mos-CHIK-VRPs at a density of 500 or 2,500 infectious units (IU)/well at the indicated temperature; and FIG. 3B is the bar chart depicting the luciferase activity in Vero cells infected with the mos-CHIK-VRPs at a density of 500 or 2,500 infectious units (IU)/well at the indicated time points. RLU, relative light unit; ns, non-significant; *, p≤0.05; **, p≤0.01.

FIGS. 4A-4B depict the use of the mos-CHIK-VRPs to assess the ability of the antibodies for neutralizing infection, in which FIGS. 4A-4B are the results for neutralizing infection of the mos-CHIK-VRPs on Vero cells with the neutralizing antibodies CHK-265, 3E7B, or 6B6C (FIG. 4A), or with the sera from CHIKV-infected patients 1-4 (FIG. 4B).

FIGS. 5A-5B depict the use of the mos-CHIK-VRPs to screen potential antiviral agents for inhibiting CHIKV infection, in which FIGS. 5A-5B are the results for suppressing infection of the mos-CHIK-VRPs on Vero cells with 6-azauridine (6-AU) (FIG. 5A), or with suramin (FIG. 5B).

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of molecular biology and virology, which are within the skill of the art. Such techniques are explained fully in the literature.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “baculoviruses” as used herein refer to arthropod-specific, double stranded DNA viruses that can be used to control insect pests. Such baculoviruses include those that infect cotton bollworm, Helicoverpa zea, tobacco budworm, Heliothis virescens, Douglas fir tussock moth, Orygia pseudotsugata, gypsy moth, Lymantria dispar, alfalfa looper, Autographa californica, European pine sawfly, Neodiiprion sertifer, and codling moth, Cydia pomonella, which are suitable as the vectors for expressing viral proteins of other arthropod-borne viruses, that is, other than baculoviruses themselves, and preferably those that are regarded as dangerous and lethal arthropod-borne viruses, such as alphaviruses.

As used herein, the term “less” refers to a “decreased” or “reduced” level or amount, usually a “statistically significant” decreased or reduced level or amount, and may include, for example, a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) relative to a reference level or amount. An decreased or reduced level or amount may also include a 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1,000-fold, 10,000-fold, or greater than 10,000-fold decrease (including all integers and ranges in between) relative to a reference level or amount. Other examples of comparisons and “statistically significant” levels or amounts are described herein. “Decrease,” as used herein, can refer to “inhibit,” “reduce,” “curb,” “abate,” “diminish,” “lessen,” or “lower.”

The term “more” refers to a “increased” or “enhanced” level or amount, usually a “statistically significant” increased or enhanced level or amount, and may include, for example, a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% increase (including all integers and ranges in between) relative to a reference level or amount. An increased or enhanced level or amount may also include a 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1,000-fold, 10,000-fold, or greater than 10,000-fold increase (including all integers and ranges in between) relative to a reference level or amount. Other examples of comparisons and “statistically significant” levels or amounts are described herein. “Increase,” as used herein, can refer to “agonize,” “enhance,” “inflate,” “escalate,” expand,” “augment,” “enlarge,” or “raise.”

The term “statistically significant” refers to the result was unlikely to have occurred by chance. Statistical significance may be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less. The term “significant” encompasses and includes the term “statistically significant.”

The term “biological sample” as used herein refers to a whole blood sample, a plasma sample, a serum sample, a saliva sample, a sputum sample, a urine sample, a mucus sample, an ascites sample, a cerebrospinal fluid sample, or an amniotic fluid sample collected from a mammal, which includes human that has or is suspected of having an infection caused by an alphavirus (e.g., CHIKV). The biological sample can be diluted or undiluted before being subject to the detection of the alphavirus VRP produced by the present recombinant baculoviruses, kits, and/or methods. In the case when an antibody against the alphavirus is present in the biological sample, the alphavirus VRP produced by the present recombinant baculovirus will specifically bind with the antibody, thereby forming a complex that is detected in an immunogenic assay (e.g., ELISA). By contrast, if the antibody against the alphavirus is not present in the biological sample, then the alphavirus VRP produced by the present recombinant baculovirus will not bind with the antibody, therefore no immunocomplex is formed.

The term “subject” or “patient” refers to an animal including the human species that is evaluable with the alphavirus VRP produced by the present recombinant baculovirus, the kit, and/or the method comprising the alphavirus VRP of the present disclosure. The term “subject” or “patient” intended to refer to both the male and female gender unless one gender is specifically indicated, and may be at any age, e.g., a child or adult. Examples of a “subject” or “patient” include, but are not limited to, a human, a rat, a mouse, a guinea pig, a monkey, a pig, a goat, a cow, a horse, a dog, a cat, a bird, and a fowl. In an exemplary embodiment, the subject is a human.

### II. Description of the Invention

The present disclosure aims at providing a recombinant baculovirus that facilitates the efficient production of an infectious but propagation-defective alphavirus VRP in mosquito cells. By use of the delicate selected promoters in the recombinant baculovirus as described herein, one may manipulate the gene expression efficiency of the recombinant baculovirus, so as to minimize cytopathic effect (CPE) in mammalian cells and maximize the production of the alphavirus VRP to the most extent.

### 1. The Recombinant Baculovirus

Accordingly, the first aspect of the present disclosure is directed to a recombinant baculovirus, which is characterized in having the ability of producing an alphavirus virus-like replicon particle (VRP) in a mosquito cell. To produce the present recombinant baculovirus, gene cassettes encoding the viral protein(s) of an alphavirus of interest are independently constructed and linked to a suitable promoter, so that a baculoviral donor vector is produced; the donor vector is then used with the baculoviral DNA to co-transfect a host cell (e.g., an insect cell, such as Sf21 cells, derived from Spodoptera frugiperda) to produce the recombinant baculovirus of the present disclosure. Specifically, the present recombinant baculovirus comprises,
a replicon comprising,
   a first promoter; and
   a first polynucleotide operably linked to the first promoter encoding at least one alphavirus non-structural protein; and
a helper comprising,
   a second promoter; and
   a second polynucleotide operably linked to the second promoter encoding at least one alphavirus non-structural protein;
      wherein,
the helper is upstream or downstream to the replicon; and
the first promoter is less efficient than the second promoter in driving gene expression in the mosquito cell.

According to some embodiments of the present disclosure, the first promoter is less efficient (<10⁻²) than the second promoter in driving gene expression in the mosquito cell; i.e., the first promoter is at least 100 times lower than the second promoter in driving gene expression in the mosquito cell.

According to embodiments of the present disclosure, the alphavirus may be a specie of Barmah Forest virus (BFV), Chikungunya virus (CHIKV), Eastern equine encephalitis virus (EEEV), Eilat virus (EILV), Everglades virus (EVEV), Middelburg virus (MIDV), O'nyong'nyong virus (ONNV), Rio Negro virus (RNV), Ross River virus (RRV), Salmon pancreas disease virus (SPDV), Semliki Forest virus (SFV), Sindbis virus (SINV), Venezuelan equine encephalitis virus (VEEV), or Western equine encephalitis virus (WEEV). In one working example, the alphavirus is CHIKV.

The baculovirus for the present recombinant baculovirus may be any kind of baculoviruses without restrictions. Non-limiting examples of such baculovirus are any of Autographa californica multiple nucleopolyhedrovirus (AcMNPV), Anagrapha falcifera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera exigua MNPV (SeMNPV), Spodoptera frugiperda MNPV (SfMNPV), or Trichoplusia ni MNPV (TnMNPV). For example, the baculovirus of AcMNPV may be used in constructing the present baculovirus.

The recombinant baculovirus of the present disclosure may produce alphavirus VRPs in mosquito cells. For example, the present recombinant baculovirus may produce alphavirus VRPs in cells derived from Aedes albopictus, Aedes aegypti, Aedes pseudoscutellaris, Anopheles sinensis, Armigeres subalbatus, Culex quinquefasciatus, Culex tritaeniorhynchus, or Toxorhynchites amboinensis. In one working example, the cells derived from Aedes pseudoscutellaris or Aedes albopictus are used to produce the alphavirus VRPs.

To make the alphavirus VRP, a replicon and a helper are required for the VRP assembly, in which said replicon comprises a first promoter and a first polynucleotide encoding at least one alphavirus non-structural protein, and said helper comprises a second promoter and a second polynucleotide encoding at least one alphavirus non-structural protein. To optimize the production of the alphavirus VRP in the mosquito cells, the promoter for driving the expression of the replicon (i.e., the first promoter) is set to be less effective than the promoter for driving the expression of the helper (i.e., the second promoter), so that the production of the replicon and the helper may keep up with each other to facilitate the alphavirus VRP assembly as much as possible, and the potential CPE in the mosquito cell as less as possible.

According to embodiments of the present disclosure, the first promoter may be a promoter of Ac5 gene, a promoter of ADH1 gene, a promoter of human 6-actin gene, a promoter of CAG gene, a promoter of CaMKIIa gene, a promoter of CaMV35S gene, a promoter of CMV gene, a promoter of EF1a gene, a promoter of GAL1 gene, a promoter of GAL10 gene, a promoter of GDS gene, a promoter of H1 gene, a promoter of PGK1 gene, a promoter of polyhedrin gene, a promoter of SV40 gene, a promoter of TEF1 gene, a promoter of TRE gene, a promoter of U6 gene, a promoter of UAS gene, a promoter of Ubc gene, or a promoter of Ubi gene. The second promoter may be at least one promoter of a promoter of the Heliothis zea Nudivirus-1 (HzNV-1) viral early expressing gene pag1, a promoter of the ceropin gene b1, a promoter of the defensin gene a4, a promoter of the heat shock protein 70 gene (hsp70), and a promoter of the homologous region 1 gene (hr1). According to one preferred embodiment of the present disclosure, the first promoter is the promoter of CMV gene, and the second promoter is a composite promoter comprising the promoter of hr1 and the promoter of pag1.

According to the embodiments of the present disclosure, the non-structural protein may be non-structural protein 1 (nsP1), non-structural protein 2 (nsP2), non-structural protein 3 (nsP3), non-structural protein 4 (nsP4), or a combination thereof. Exemplary combination of the non-structural protein suitable for use in the present disclosure includes, but is not limited to, the combination of nsP1 and nsP2; nsP1 and nsP3; nsP1 and nsP4; nsP2 and nsP3; nsP2 and nsP4; nsP3 and nsP4; the combination of nsP1, nsP2, and nsP3; nsP1, nsP2, and nsP4; nsP1, nsP3, and nsP4; nsP2, nsP3, and nsP4; or the combination of nsP1, nsP2, nsP3, and nsP4. In one preferred embodiment, the alphavirus non-structural protein used to produce the alphavirus VRP comprises the combination of nsP1, nsP2, nsP3, and nsP4.

According to the embodiments of the present disclosure, the structural protein is capsid (C), envelope 3 (E3), envelope 2 (E2), 6K protein (6K), envelope 1 (E1), or a combination thereof. Exemplary combination of the structural protein may be any of the combination of C and E3; C and E2; C and 6K; C and E1; E3 and E2; E3 and 6K; E3 and E1; E2 and 6K; E2 and E1; 6K and E1; the combination of C, E3, and E2; C, E3, and 6K; C, E3, and E1; C, E2, and 6K; C, E2, and E1; C, 6K, and E1; E3, E2, and 6K; E3, E2, and E1; E3, 6K, and E1; E2, 6K, and E1; the combination of C, E3, E2, and 6K; C, E3, E2, and E1; C, E3, 6K, and E1; C, E2, K6, and E1; E3, E2, 6K, and E1; or the combination of C, E3, E2, 6K, and E1. In one preferred embodiment, the alphavirus structural protein used to produce the alphavirus VRP comprises the combination of C, E3, E2, 6K, and E1.

According to preferred embodiments of the present disclosure, the thus produced recombinant baculoviral donor vector is then placed into a Bac-to-Bac baculovirus expression system to generate a recombinant baculoviruse bacmid DNA by site-specific transposition in a competent E. coli cell having a recombinant bacmid with the necessary viral backbone therein, which contains the propagation-essential genes. The recombinant baculoviruse bacmid DNA in the insect host cell allows the generation of a recombinant baculovirus, which is capable of propagating in the mosquito host cell. Suitable mosquito host cell that may be used in the present disclosure is as described above.

The recombinant baculovirus may be further detected, selected, and purified, such as by following the expression of an exogenous gene, i.e., a reporter protein, an expression tag, or a combination thereof. To this end, the exogenous gene is operably linked to a subgenomic promoter that is operably linked to the first polynucleotide in the baculoviral vectors. Examples of reporter protein include, but are not limited to, blue fluorescence protein (BFP), cyan fluorescent protein (CFP), green fluorescence protein (GFP), enhanced green fluorescence protein (eGFP), Discosoma sp. red fluorescent protein (DsRed), yellow fluorescent proteins (YFP), enhanced yellow fluorescent proteins (eYFP), Anemonia majano fluorescent protein (amFP), Clavularia fluorescent protein (cFP), Discosoma fluorescent protein (dsFP), Zoanthus fluorescent protein (zFP), β-galactosidase (lacZ), chloramphenicol acetyltransferase (CAT), luciferase (Luc), β-lactamase, aminoglycoside-3′-phosphotransferase (APH(3′)), orotidine-5′-phosphate decarboxylase (ODCase), chitin binding protein (CBP), maltose binding protein (MBP), glutathione-S-transferase (GST), thioredoxin (TRX), alkaline phosphatase (AP), biotin-carboxy carrier protein (BCCP), and calmodulin binding peptide (CBP). In addition, examples of the expression tag are FLAG-tag, hemagglutinin (HA)-tag, poly-histidine tag, MYC-tag, NE-tag, Spot-tag, Strep-tag, or bacteriophage T7 epitope (T7-tag). In some preferred embodiments of the present disclosure, the reporter protein is eGFP or Luc. It should be noted that the reporter protein (e.g., eGFP or Luc) is not a necessary feature for the aim of this invention, which is, detecting an anti-alphavirus antibody that might be present in a biological sample or screening an antivital agent for treating an alphavirus infection.

Depending on different purposes, the exogenous gene may encode any proteins of interest, such as an immunogen for preventing a pathogen infection or cancer. In this regard, the alphavirus VRP produced from the present recombinant baculovirus may serve as a vector for delivering the exogenous proteins.

The thus produced recombinant baculovirus may be collected to transduce the mosquito cell to express the replicon (i.e., non-structural protein(s)) and the helper (i.e., structural protein(s)) of the alphavirus, so that the alphavirus VRP is then assembled in the transduced mosquito cell. As such, the alphavirus VRP produced by the present recombinant baculovirus and the use to make a vaccine for preventing alphavirus infection, or preventing a pathogen infection other than alphavirus or cancer (depending on the exogenous genes used in the present recombinant baculovirus), are within the scope of the present disclosure.

### 2. The Method for Detecting an Antibody Against the Alphavirus

The recombinant baculovirus constructed in accordance with the methods described above may produce an alphavirus VRP, accordingly, the alphavirus VRP may be used as antigens for capturing antibodies of the interested alphavirus, if any, in a biological sample.

Thus, another aspect of the present disclosure aims at providing a method for detecting an antibody against an alphavirus in a biological sample (hereafter, “the detecting method”). The method includes steps of:
(a) mixing the biological sample with an alphavirus VRP produced by the present recombinant baculovirus; and
(b) detecting a complex formed between the alphavirus VRP and the antibody against the alphavirus in the biological sample in an immunological assay.

In one preferred embodiment, a serum sample of a human subject suspected of having a CHIKV infection is mixed with the alphavirus VRP produced by the present recombinant baculovirus; preferably, the alphavirus VRP is a CHIK-VRP, so as to detect an anti-CHIKV antibody (preferably, a neutralization antibody) in the serum sample. Accordingly, if the human subject was indeed infected with CHIKV, then the antibodies (i.e., autoantibodies against CHIK virus) in the serum will bind the CHIK-VRP and form a complex.

Exemplary immunological assays suitable for detecting the antigen-antibody complex formed in the present method include, but are not limited to, radioimmunoassay, enzyme-linked immunosorbent assay (ELISA), “sandwich” immunoassay, in situ immunoassays (e.g., using colloidal gold, enzyme, or radioisotope labels), dot blot, agglutination assay (e.g., gel agglutination assay, hemagglutination assay, and etc.), complement fixation assay, immunofluorescence assay, and immunoelectrophoresis assay, and etc. In one embodiment, the formed complex is detected by use of ELISA.

According to embodiments of the present disclosure, antibodies are detected in bodily fluids, such as whole blood, plasma, serum, saliva, sputum, urine, mucus, ascites, cerebrospinal fluid, amniotic fluid, and purified or filtered forms thereof. In other embodiments, antibodies are detected from a serum sample.

### 3. The Method for Screening an Antiviral Agent Against the Alphavirus

Further, the alphavirus VRP produced by the present recombinant baculovirus may also be used as a platform for screening an antiviral agent suitable for treating an alphavirus infection. Thus, a further aspect of the present disclosure is meant to provide a method to this end. Said method comprises steps of,
(a) mixing a drug candidate with an alphavirus VRP produced by the present recombinant baculovirus of claim 1 to form a mixture; and
(b) applying the mixture of step (a) to a mammalian cell.
   wherein,
the drug candidate is the antiviral agent if the amount of the alphavirus VRP present in the mammalian cell of step (b) is lower than that of a control, which is the mammalian cell treated with the alphavirus VRP produced by the present recombinant baculovirus alone.

According to embodiments of the present disclosure, said antiviral agent may be a molecule of any kind, such as an antibody (e.g., a neutralizing antibody), a protein, a peptide derived from a protein by digestion or other fragmentation techniques, a peptidomimetic (e.g., a peptoid), a small molecule (such as a hormone, a metabolite, a drug, a drug metabolite), a nucleic acid (DNA, RNA, or a fragment thereof produced by an enzymatic, a chemical, or other fragmentation processes). The term “small molecule” or “metabolite” means a multi-atom molecule other than proteins, peptides, and DNA; the term can include, but is not limited to, amino acids, amino acid analogs, polynucleotides, polynucleotide analogs, nucleotides, nucleotide analogs, steroid and other small hormones, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Accordingly, the antiviral agent is able to inhibit the infection of the alphavirus on the mammalian cell; preferably, said alphavirus is CHIKV. Where the antiviral agent is an antibody, the present screening method may further be used to evaluate the ability of the antibody for neutralizing the infection of the alphavirus (e.g., CHIKV) on the mammalian cell. According to some embodiments of the present disclosure, the antiviral agent is a small molecule: 6-azauridine (6-AU) or suramin.

As used herein, exemplary mammalian cells include, but are not limited to, human cells, primate cells, rodent cells (e.g., mouse and rat cells), and canine cells. Mammalian cell lines for use in accordance with the present disclosure include, without limitation, 293-T, 3T3 cells, 4T1, 721, 9L, A-549, A172, A20, A253, A2780, A2780ADR, A2780cis, A431, ALC, B16, B35, BCP-1 cells, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, HEK-293, HeLa, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, Ma-Mel 2, Ma-Mel 3, Ma-Mel 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, 20) NIH-3T3, NW-145, OPCN/OPCT cell lines, Peer, PNT-1A/PNT 2, PTK2, Raji, RBL cells, RenCa, RIN-5F, RMA/RMAS, S2, Saos-2 cells, SiHa, SKBR3, SKOV-3, T-47D, T2, T84, THP1 cell line, U373, U87, U937, VCaP, Vero cells, WM39, WT-49, X63, YAC-1 and YAR cells. In one working example, the mammalian cell used in the present screening method is Vero cells.

The method for evaluating the amount of the alphavirus VRP present in the mammalian cell (i.e., evaluating virus neutralization or inhibition in the infected cells) is well known in the art, for example, a plaque reduction test or a serum virus neutralization assay may be used for the above purpose. Alternatively, in the case that the alphavirus VRP has a fluorescent reporter protein, the infected cell may be directly examined under a microscope.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Materials and Methods

### 1. Generation of the Recombinant Baculovirus

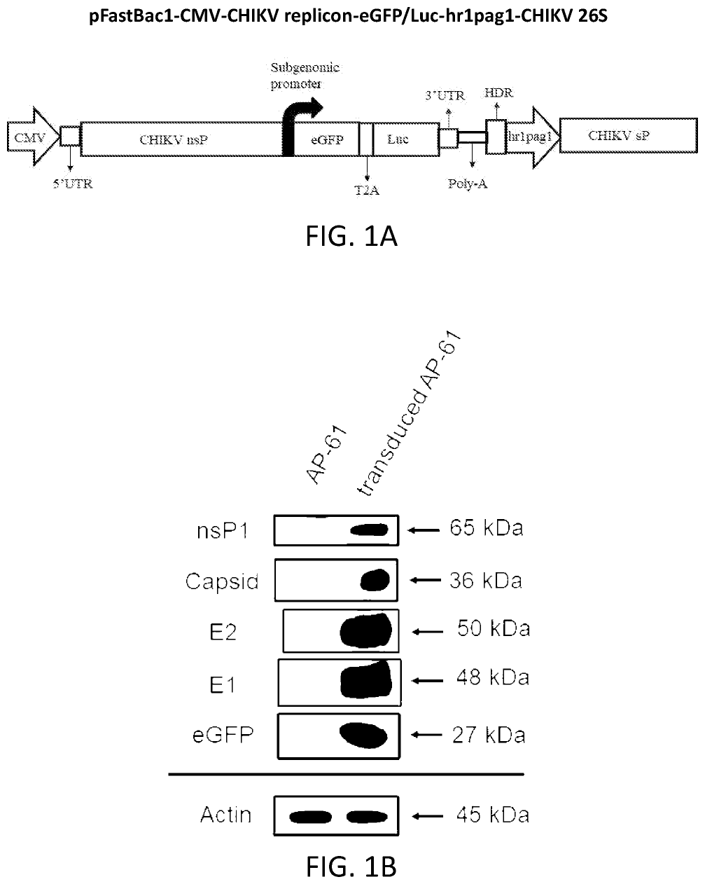

The donor vector of the recombinant baculovirus was constructed on the plasmid pFastBac1 of an AcMNPV-based backbone, comprising in sequence from 5′ to 3′: the promoter of CMV gene, the CHIKV replicon (containing nsP1-nsP4 proteins and the subgenomic promoter), the eGFP-Thosea asigna virus 2A self-cleaving peptide (T2A)-Luc (eGFP-T2A-Luc) gene cassette, the polyA signal, the hepatitis delta virus ribozyme (HDR) gene cassette, the promoters of hr1pag1, and the CHIKV 26S (containing C, E3, E2, 6K, and E1 proteins; from CHIKV OPY-1 strain, GenBank: KT449801), as shown in FIG. 1A, in which the hr1pag1-CHIKV 26S gene cassette was subcloned into pFastBac1-VSVG-CHIKV replicon-GFP in tandem by using the pair of primers having the nucleic acid sequences of SEQ ID NOs. 1-2, and the GFP gene therein was replaced with the eGFP-T2A-Luc gene cassette. Table 1 summarizes the primers used in the present study. The resulting plasmid was termed as “pFastBac1-CMV-CHIKV replicon-eGFP/Luc-hr1pag1-CHIKV 26S” (FIG. 1A). The thus generated donor vector of recombinant baculovirus was used to produce recombinant baculoviruses in accordance with the manufacturer's instructions (the Bac-to-Bac baculovirus expression system; Invitrogen, USA). The viral titer of the produced recombinant baculovirus was determined with a commercially available assay kit (the BacPAK™ baculovirus rapid titer kit; Takara, USA), with the following modifications: The fixed cells were permeabilized with 0.5% Triton X-100 in PBS for 5 minutes prior to immunostaining with a monoclonal antibody against CHIKV capsid (Native Antigen Company) to replace anti-GP64 antibodies.

TABLE 1
Primers used in the present study

| SEQ ID NO. | Name | Type | Sequence (from 5′ to 3′) |
|---|---|---|---|
| 1 | Cloning-F | DNA | TGGCTAAGGGAGGGCGGTTTGTGTTTTACAAG |
| | primer | | TAGAATTCTACC |
| 2 | Cloning-R | DNA | CTTGGTACCGCATGCTGTTTCTCGAGCGGCCGC |
| | primer | | CCCG |
| 3 | Actin-F | DNA | GGCCAGGTCATCACCATT |
| | primer | | |
| 4 | Actin-R | DNA | ATGTCCACGTCACACTTCATG |
| | primer | | |
| 5 | 3′UTR-F | DNA | CAAACGGAAGAGATGTAGG |
| | primer | | |
| 6 | 3′UTR-R | DNA | GGGTTCGGAGAATCGTGG |
| | primer | | |

F: forward; R: reverse.

### 2. Cells

AP-61 cells (derived from Aedes pseudoscutellaris) were cultured in Leibovitz L-15 medium (Gibco) supplemented with 10% fetal bovine serum (FBS) (Gibco) and 1% antibiotic-antimycotic (Gibco) at 28° C. C6/36 cells (derived from Aedes albopictus) (ATCC® CRL-1660™) were cultured in RPMI 1640 medium (Gibco) supplemented with 10% FBS (Gibco) and 1% penicillin/streptomycin (Gibco) at 28° C. under 5% CO₂. Vero cells (ATCC® CCL-81™) were cultured in minimum essential media (MEM) (Gibco) containing 10% FBS (Gibco), 1% L-glutamine (Gibco), 1% MEM non-essential amino acids (NEAA) (Gibco), 1% sodium pyruvate (Gibco), and 1% penicillin/streptomycin (Gibco) at 37° C. under 5% CO₂.

### 3. Production and Titration of Mos-CHIK-VRPs

For preparing the mos-CHIK-VRPs, AP-61 cells or C6/36 cells were seeded in T-25 flasks (2×10⁶ cells/flask), incubated overnight, and transduced with the recombinant baculovirus at a multiplicity of infection (MOI) of 20 in a growth medium supplemented with 5% FBS. At 24 hours post-transduction (hpt), the growth medium was refreshed, and the cells were culture for another 3-4 days. Half of the culture supernatant was then carefully harvested, and fresh growth media were added to the culture for another 3-4 days; and the process was repeated once more. The collected supernatants were centrifuged at 4,000×g for 20 minutes at 4° C., and filtered with a 0.22 μM filter, before subjected to aliquote and stored at −80° C.

For titration, Vero cells were seeded at 1.5×10⁴ cells/well (96-well plate) one day before commencing the experiment. The VRP stock was diluted with MEM in a 10-fold serial dilution, whereupon the aliquot of the dilution was added to cultured cells for 1 hour at 28° C., whereupon the medium was refreshed, and the incubation continued for another 20 hour. The number of eGFP-positive cells was counted under an inverted fluorescence microscope (Leica Microsystems).

### 4. Western Blot Analysis

The transduced AP-61 cells were prepared as described above. For the preparation of infected Vero cells, Vero cells (2×10⁵ cells/well/6-well plate) were infected with mos-CHIK-VRPs at an MOI of 0.05 for 1 hour at 28° C., and then cultured for another 20 hours at 28° C. The cells were collected and lysed in RIPA buffer (Thermo Fisher Scientific), and the resulting protein samples were separated on a 4 to 12% Bis-Tris gel (Invitrogen), before being transferred onto nitrocellulose membranes. The membranes were incubated with primary antibodies of rabbit anti-CHIKV nsP1 polyclonal antibody (1:5,000) (GeneTex), anti-CHIKV E1 polyclonal antibody (1:1,000), anti-CHIKV E2 monoclonal antibody (mAb) (1:1,000) (1b1, from Taiwan CDC), mouse anti-GFP mAb (1:1,000) (Santa Cruz Biotechnology), mouse anti-CHIKV capsid mAb (1:1,000) (Native Antigen Company), or mouse anti-actin mAb (1:5,000) (Sigma Aldrich); and then with secondary antibodies of goat anti-mouse or goat anti-rabbit secondary antibodies conjugated with horseradish peroxidase (HRP) (Sigma Aldrich). The signals were visualized after incubating the membranes with an ECL substrate, and were captured using an imaging system (Amersham Imager 600; GE Healthcare).

### 5. Quantification of Viral RNA

Vero cells were seeded at a density of 1×10⁵ cells/well (24-well plate) and infected with the mos-CHIK-VRPs at an MOI of 0.1. The infected cells were subjected to total cellular RNA extraction at 1 or 24 hpi by using an RNA miniprep kit (New England Biolabs), and then subjected to reverse transcription with a reverse transcription kit (Qiagen). Quantitative RT-PCR (qRT-PCR) was carried out by a real-time PCR System (Applied Biosystems) in triplicate, using the primers as provided in Table 1 respectively having the nucleic acid sequences of SEQ ID NOs. 3-4 for actin and SEQ ID NOs. 5-6 for 3′UTR, in which the 3′UTR expression was estimated based on the relative quantification by the comparative critical threshold (CT) method. The level of the viral RNA expression was normalized to the actin of the endogenous control. Fold changes were calculated by the ddCt method, in which the data obtained at 1 hpi was set as a baseline.

### 6. Luciferase Assay

Luciferase activity in the mos-CHIK-VRPs infected Vero cells was measured using a commercial luciferase assay kit (Promega) in accordance with the manufacturer's instructions. Signals reflecting the luciferase activity was read by a microplate reader (BioTek).

### 7. VRP-Based Neutralization Assay

Vero cells were seeded at a density of 1.5×10⁴ cells/well (96-well plate). For eGFP detection, Vero cells were infected with the VRPs that had been pre-incubated with 50-fold diluted sera from patient or normal individual for 1 hour at 37° C. and 1 hour at 28° C., and then the infected Vero cells continued incubation for another 20 hour at 28° C. The signals of the eGFP-expressing cells were detected by an inverted fluorescence microscope (Leica Microsystems).

For luciferase assay, VRPs were independently incubated with serial dilutions of the CHIKV neutralizing mAbs (CHK-265 (Absolute Biotech) and 3E7B (Novus Biologicals)) and the flavivirus neutralizing mAb (6B6C (Bio-Rad), as a negative control); sera from patients and from healthy individuals (as a negative control); sera from rabbits immunized with inactivated CHIKV and from pre-immune rabbits (as a negative control) for 1 hour at 37° C. Then, Vero cells were infected with the foregoing VRPs (2500 IU/well) for 1 hour at 34ºC, whereupon the medium was refreshed and the infected Vero cells continued incubation for another 5 hours at 34° C., before being subjected to a luciferase assay.

### 8. VRP-Based Antiviral Drug Assay

Suramin and 6-azauridine (6-AU) (both from Sigma-Aldrich) were dissolved in water to prepare the stock solutions (suramin, 50 mg/ml, 35 mM; 6-AU, 50 mg/ml, 200 mM) and then stored at −20° C. until use. Vero cells were seeded at a density of 1.5×10⁴ cells/well (96 well white/clear bottom plate), and then infected with 2,500 IU VRPs/well in the presence of serially diluted suramin or 6-AU in MEM for 1 hour at 34° C. The suramin- or 6-AU-treated cells were then grown in a 5% FBS medium containing the corresponding concentrations of suramin or 6-AU for another 5 hours at 34° C., then VRP activity was measured by luciferase assay.

### 9. Statistics

All the experiments were conducted in triplicate. Data were presented as mean±standard deviation (SD). Statistical significance was analyzed using the Student's t-test. For all statistical analyses, a two-tailed p-value≤0.05 was considered significant. Calculations and diagrams were generated using the software GraphPad Prims 6.01.

### Example 1 Design and Characterization of the Recombinant Baculovirus

In this example, a recombinant baculovirus is constructed to comprise gene cassettes as depicted in FIG. 1A in accordance with the method as described in the section of “Materials and Methods,” which bears a CMV promoter-driven CHIKV replicon (comprising the CHIKV nsP1-4) having eGFP and Luc under the transcription control of CHIKV 26S subgenomic promoter, and a hr1pag1 promoter-driven CHIKV 26S gene (the CHIKV helper, comprising CHIKV sPs). It was noticed that the CMV promoter was less efficient (<10⁻²) in driving the expression of the CHIKV replicon than the hr1pag1 promoter in driving the expression of the CHIKV helper in the mosquito cells (data not shown).

The thus generated recombinant baculovirus was analyzed by Western blot to confirm the expression of the nsP1, the capsid, E2, E1 and the eGFP in the recombinant baculovirus-transduced AP-61 cells. Results are provided in FIG. 1B, in which the signals of the above proteins were detected in the recombinant baculovirus-transduced AP-61 cells alone, but not in the mock control AP-61 cells, suggesting the recombinant baculovirus was successfully produced and were able to express the viral components in the transduced AP-61 cells. Taken together, these results evidenced that the recombinant baculovirus was successfully produced and could express all the necessary viral components in the transduced AP-61 cells.

### Example 2 Production and Characterization of the Mos-CHIK-VRPs

In this example, whether the CHIK-VRPs may be successfully produced in the mosquito cells (the mos-CHIK-VRPs) after the cells transduced with the recombinant baculovirus of Example 1; and whether the resulting mos-CHIK-VRPs may infect mammalian cells (e.g., Vero cells) were investigated.

To afore-mentioned purposes, the mos-CHIK-VRPs were produced in accordance with procedures set forth in the section of “Materials and Methods;” and no noticeable cell death was found in the mosquito cells that produced the mos-CHIK-VRPs, that is, no visible CPE was noticed in those cells (data not shown). The generated mos-CHIK-VRPs were then tested for their ability to infect the mammalian cells.

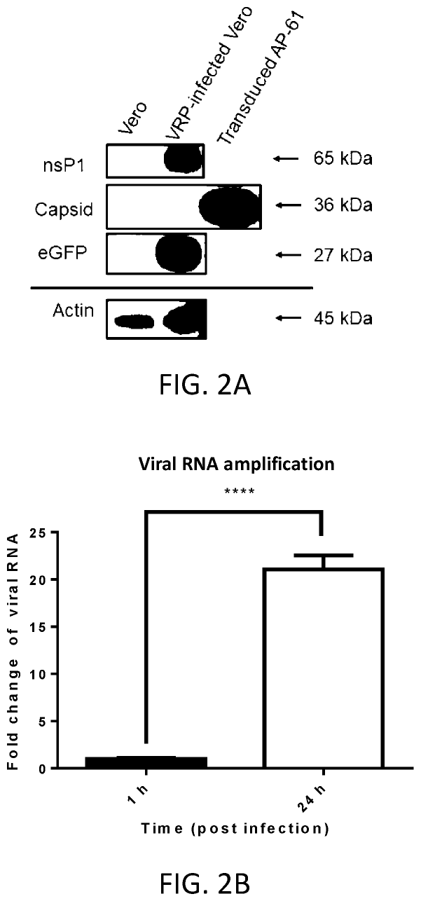

Specifically, the culture media from either Ap-61 or C6/36 cells transduced with the recombinant baculovirus of Example 1 (containing the mos-CHIK-VRPs) were used to infect Vero cells to evaluate if the mos-CHIK-VRPs possess the ability to infect mammalian cells. The production of the mos-CHIK-VRPs was confirmed by Western blot analysis to confirm the expression of the nsP1, the capsid, and the eGFP in the mos-CHIK-VRP-infected Vero cells. As shown in FIG. 2A, the signals specific to the nsP1 and the eGFP, but not to the capsid, were detected in infected Vero cells, suggesting the mos-CHIK-VRPs were successfully produced and could express the viral components in the infected Vero cells, except the capsid protein. In addition, there was no noticeable cell death (i.e., no CPE) in the infected cells (data not shown).

The activity of the mos-CHIK-VRPs in the mos-CHIK-VRP-infected cells was assessed via evaluating the viral replication in the infected cells. To this purpose, the total viral RNA from VRP-infected cells harvested at either 1 or 24 hpi was determined by qRT-PCR. It is found that the viral RNA level in the infected Vero cells at 24 hpi elevated significantly (FIG. 2B), indicating the mos-CHIK-VRPs were highly active in the infected cells. Taken together, these results evidenced that the mos-CHIK-VRPs were successfully produced, which were infectious to and active in the mammalian cells.

### Example 3 the Kinetics of the Mos-CHIK-VRPs

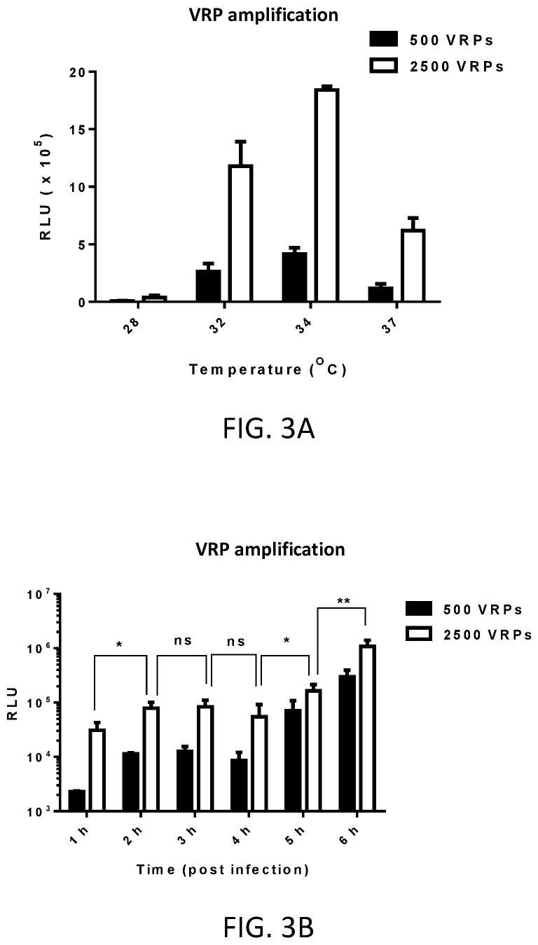

The effect of temperature and the amount of the VRP on the activity of the VRP in the VRP-infected cells was assessed herein. Vero cells were infected with VRPs at a density of 500 or 2,500 infectious units (IU)/well for 6 hours at 28, 32, 34, or 37° C.; and the activity of the VRP in the VRP-infected cells was determined by luciferase assay. Significant levels of VRP activity were observed in cells infected with 2,500 IU of VRPs at 32° ° C., 34° C., or 37° C., while cells at 28° C. gave negligible level of VRP activity (FIG. 3A). Similar trend was also found for cells infected with 500 IU of VRPs at 32° C., 34° C., 37° C., or 28° C. (FIG. 3A).

Further, the VRP activity in the VRP-infected Vero cells was tracked during the period of 1-6 hpi at 34° C., and results are provided in FIG. 3B. It was found that VRP activity in cells (either infected with 500 or 2,500 IU VRPs) started to increase significantly as early as 5-6 hpi (FIG. 3B). The activity of the VRP was higher in cells infected with 2,500 IU VRPs than those infected with 500 IU VRPs in all the treatment groups (FIGS. 3A-3B). No perceivable CPE was found in all the VRP-infected cells. Taken together, these data confirmed that the VRP activity was affected by both temperature and the level of VRP used during infection.

### Example 4 the Ability of Mos-CHIK-VRPs in Assessing CHIKV Antibodies or Screening Anti-CHIKV Agent

### 4.1 CHIKV Antibodies

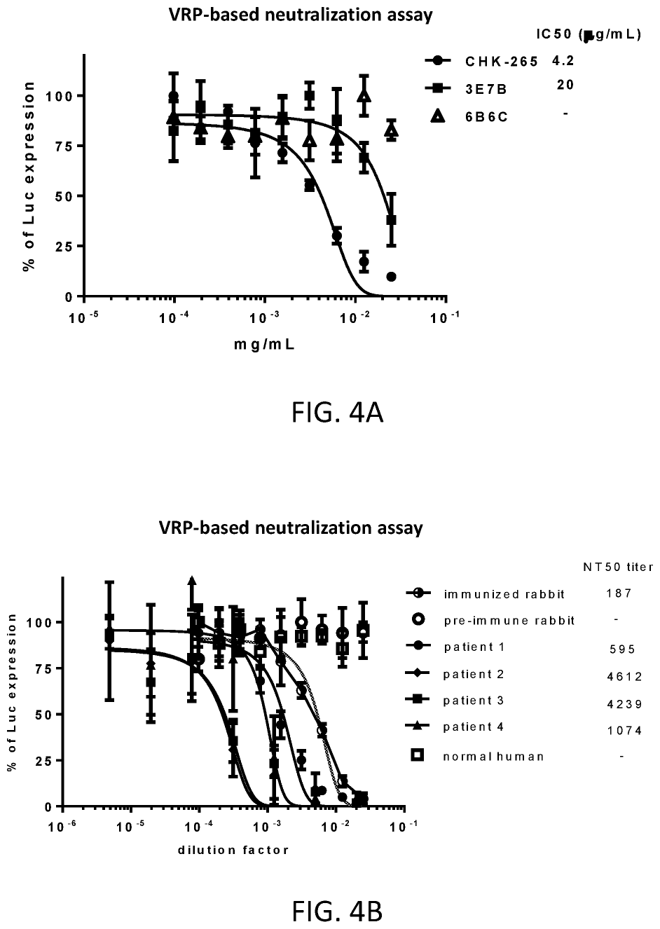

The mos-CHIK-VRPs were analyzed for their abilities in assessing CHIKV antibodies, including two commercial monoclonal antibodies of CHIKV and antibodies present in the sera harvested from CHIKV patients. Results are provided in FIGS. 4A-4B.

It was found that the mos-CHIK-VRPs could be successfully used in assessing the ability of mAbs of CHK265 or 3E7B in neutralizing CHIKV with IC₅₀ of 4.2 μg/ml or 20 μg/ml (FIG. 4A), as well as antibodies in the sera harvested from CHIKV patients (FIG. 4B). No neutralization activity was detected from mock treatment groups, including the 6B6C mAb and the normal sera (i.e., serum from a pre-immune rabbit or one normal individual) treatment groups.

### 4.2 Anti-CHIKV Agent

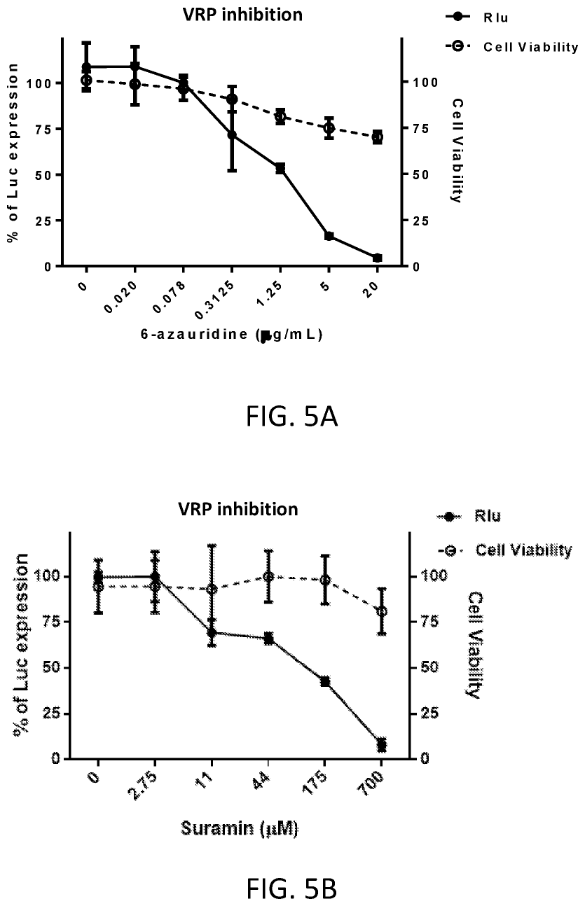

In this example, 6-AU (a viral replication inhibitor) and suramin (a viral-entry inhibitor) were respectively tested for its ability in suppressing CHIKV by co-incubating with cells infected with the present mos-CHIK-VRPs. Results are provided in FIGS. 5A-5B.

As shown in FIGS. 5A-5B, a concentration-dependent inhibition of VRP infection was observed within a few hours, with IC₅₀ value of 1.286 μg/ml in 6-AU treated cells, and 81.3 μM in suramin treated cells. Taken together, these results confirmed that the mos-CHIK-VRP could be used for evaluating agents that suppressed CHIKV, such agents are candidate agents for the development of medicaments suitable for treating CHIKV infection.

In sum, the present invention in essence provides a recombinant baculovirus capable of producing the CHIK-VRP in mosquito cells, thus eliminating the need of biosafety level 3 (BSL-3) laboratory in handling CHIKV, and the CHIK-VRP thus produced are useful for the manufacture of vaccines against CHIKV infection, agents for detecting CHIKV antibodies, or screening compounds for treating CHIKV infection, and etc.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant baculovirus capable of producing an alphavirus virus-like replicon particle (VRP) in a mosquito cell, comprising,
a replicon comprising,
a first promoter; and
a first polynucleotide operably linked to the first promoter and encodes at least one alphavirus non-structural protein; and
a helper comprising,
a second promoter; and
a second polynucleotide operably linked to the second promoter and encodes at least one alphavirus structural protein;
wherein,
the helper is upstream or downstream to the replicon; and
the first promoter is less efficient than the second promoter in driving gene expression in the mosquito cell.

2. The recombinant baculovirus of claim 1, wherein the first promoter is selected from the group consisting of a promoter of Ac5 gene, a promoter of ADH1 gene, a promoter of human 6-actin gene, a promoter of CAG gene, a promoter of CaMKIIa gene, a promoter of CaMV35S gene, a promoter of CMV gene, a promoter of EF1a gene, a promoter of GAL1 gene, a promoter of GAL10 gene, a promoter of GDS gene, a promoter of H1 gene, a promoter of PGK1 gene, a promoter of polyhedrin gene, a promoter of SV40 gene, a promoter of TEF1 gene, a promoter of TRE gene, a promoter of U6 gene, a promoter of UAS gene, a promoter of Ubc gene, and a promoter of Ubi gene.

3. The recombinant baculovirus of claim 2, wherein the first promoter is the promoter of CMV gene.

4. The recombinant baculovirus of claim 1, wherein the second promoter is at least one promoter selected from the group consisting of a promoter of the Heliothis zea Nudivirus-1 (HzNV-1) viral early expressing gene pag1, a promoter of the ceropin gene b1, a promoter of the defensin gene a4, a promoter of the heat shock protein 70 gene (hsp70), and a promoter of the homologous region 1 gene (hr1).

5. The recombinant baculovirus of claim 4, wherein the second promoter comprises the promoter of hr1 and the promoter of pag1.

6. The recombinant baculovirus of claim 1, wherein the at least one alphavirus non-structural protein is selected from the group consisting of non-structural protein 1 (nsP1), non-structural protein 2 (nsP2), non-structural protein 3 (nsP3), non-structural protein 4 (nsP4), and a combination thereof.

7. The recombinant baculovirus of claim 1, wherein the at least one alphavirus structural protein is selected from the group consisting of capsid (C), envelope 3 (E3), envelope 2 (E2), 6K protein (6K), envelope 1 (E1), and a combination thereof.

8. The recombinant baculovirus of claim 1, further comprising,
a subgenomic promoter operably linked to the first polynucleotide; and
an exogenous gene operably linked to the subgenomic promoter.

9. The recombinant baculovirus of claim 8, wherein the exogenous gene encodes a reporter protein, an expression tag, or a combination thereof.

10. The recombinant baculovirus of claim 9, wherein the reporter protein is selected from the group consisting of, blue fluorescence protein (BFP), cyan fluorescent protein (CFP), green fluorescence protein (GFP), enhanced green fluorescence protein (eGFP), Discosoma sp. red fluorescent protein (DsRed), yellow fluorescent proteins (YFP), enhanced yellow fluorescent proteins (eYFP), Anemonia majano fluorescent protein (amFP), Clavularia fluorescent protein (cFP), Discosoma fluorescent protein (dsFP), Zoanthus fluorescent protein (zFP), β-galactosidase (lacZ), chloramphenicol acetyltransferase (CAT), luciferase (Luc), β-lactamase, aminoglycoside-3′-phosphotransferase (APH(3′)), orotidine-5′-phosphate decarboxylase (ODCase), chitin binding protein (CBP), maltose binding protein (MBP), glutathione-S-transferase (GST), thioredoxin (TRX), alkaline phosphatase (AP), biotin-carboxy carrier protein (BCCP), and calmodulin binding peptide (CBP).

11. The recombinant baculovirus of claim 9, wherein the expression tag is FLAG-tag, hemagglutinin (HA)-tag, poly-histidine tag, MYC-tag, NE-tag, Spot-tag, Strep-tag, or bacteriophage T7 epitope (T7-tag).

12. The recombinant baculovirus of claim 1, wherein the baculovirus is any of Autographa californica multiple nucleopolyhedrovirus (AcMNPV), Anagrapha falcifera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera exigua MNPV (SeMNPV), Spodoptera frugiperda MNPV (SfMNPV), or Trichoplusia ni MNPV (TnMNPV).

13. The recombinant baculovirus of claim 12, wherein the baculovirus is AcMNPV.

14. The recombinant baculovirus of claim 1, wherein the alphavirus is selected from the group consisting of Barmah Forest virus (BFV), Chikungunya virus (CHIKV), Eastern equine encephalitis virus (EEEV), Eilat virus (EILV), Everglades virus (EVEV), Middelburg virus (MIDV), O'nyong'nyong virus (ONNV), Rio Negro virus (RNV), Ross River virus (RRV), Salmon pancreas disease virus (SPDV), Semliki Forest virus (SFV), Sindbis virus (SINV), Venezuelan equine encephalitis virus (VEEV), and Western equine encephalitis virus (WEEV).

15. The recombinant baculovirus of claim 14, wherein the alphavirus is CHIKV.

16. The recombinant baculovirus of claim 1, wherein the mosquito cell is a cell derived from Aedes albopictus, Aedes aegypti, Aedes pseudoscutellaris, Anopheles sinensis, Armigeres subalbatus, Culex quinquefasciatus, Culex tritaeniorhynchus, or Toxorhynchites amboinensis.

17. The recombinant baculovirus of claim 16, wherein the mosquito cell is the cell derived from Aedes pseudoscutellaris or Aedes albopictus.

18. A method for detecting an antibody against an alphavirus in a biological sample, comprising,
(a) mixing the biological sample with an alphavirus VRP produced by the recombinant baculovirus of claim 1; and
(b) detecting a complex formed between the alphavirus VRP and the antibody against the alphavirus in the biological sample in an immunological assay,
wherein,
the formation of the complex indicates the presence of the antibody against the alphavirus in the biological sample.

19. The method of claim 18, wherein the alphavirus is CHIKV.

20. A method for screening an antiviral agent suitable for treating an alphavirus infection, comprising,
(a) mixing a drug candidate with an alphavirus VRP produced by the recombinant baculovirus of claim 1 to form a mixture; and
(b) applying the mixture of step (a) to a mammalian cell,
wherein,
the drug candidate is the antiviral agent if the amount of the alphavirus VRP present in the mammalian cell of step (b) is lower than that of a control, which is the mammalian cell treated with the alphavirus VRP produced by the recombinant baculovirus of claim 1 alone.


SEQUENCE LISTING (6 sequences)

SEQ ID NO: 1 - DNA, 44 bp, synthetic construct
  source 1..44: mol_type = other DNA
tggctaagggagggcggtttgtgttttacaagtagaattctacc

SEQ ID NO: 2 - DNA, 37 bp, synthetic construct
  source 1..37: mol_type = other DNA
cttggtaccgcatgctgtttctcgagcggccgccccg

SEQ ID NO: 3 - DNA, 18 bp, synthetic construct
  source 1..18: mol_type = other DNA
ggccaggtcatcaccatt

SEQ ID NO: 4 - DNA, 21 bp, synthetic construct
  source 1..21: mol_type = other DNA
atgtccacgtcacacttcatg

SEQ ID NO: 5 - DNA, 19 bp, synthetic construct
  source 1..19: mol_type = other DNA
caaacggaagagatgtagg

SEQ ID NO: 6 - DNA, 18 bp, synthetic construct
  source 1..18: mol_type = other DNA
gggttcggagaatcgtgg